(19) United States
(12) Patent Application Publication
(10) Pub. No.: US 2005/0277183 A1
(43) Pub. Date: Dec. 15, 2005

(54) Electroporation cuvette

(76) Inventor: Ronald Lee, Eaton, OH (US)

Correspondence Address: DINSMORE & SHOHL LLP;One Dayton Centre, Suite 1300, One South Main Street, Dayton, OH, 45402-2023, US

(21) Appl. No.: 11/131,871

(22) Filed: May 18, 2005

Related U.S. Application Data
(60) Provisional application No. 60/572,309, filed on May 18, 2004.

(51) Int. Cl. C12M 1/42

(52) U.S. Cl. 435/285.2

(57) ABSTRACT

Cuvettes and methods of using cuvettes in electroporation are provided. The cuvette comprises a cuvette body, and an opening, wherein the cuvette body comprises an electrode including a pair of parallel spaced electrode plates, a cavity and a well disposed inside the electrode plates. The cuvette is configured to produce an electric field between the electrode plates; wherein the electric field is operable to create pores in a cell sample present in the well.

### CROSS REFERENCE TO RELATED APPLICATION

This patent application claims the benefit of U.S. Provisional Application Ser. No. 60/572,309, filed May 18, 2004.

### FIELD OF THE INVENTION

The present invention relates generally to cuvettes and specifically to electroporation cuvettes designed to accommodate well volumes below 100 μL.

### BACKGROUND OF THE INVENTION

Cuvettes have become a widely used medium in the electroporation field. Electroporation describes the electro-cell manipulation method where electrical fields are used to create pores in cells without causing permanent damage to them. Electroporation was further developed to aid in the insertion of various molecules into cell cytoplasm by temporarily creating pores in the cells through which the molecules pass into the cell. Electroporation has enabled implant materials, such as DNA, genes, and various chemical agents, to be inserted into many different types of cells. As advances in electroporation are made, the need arises for improvements in components thereof, including cuvettes.

### SUMMARY OF THE INVENTION

In accordance with one embodiment, a cuvette comprising a cuvette body, and an opening is provided. The cuvette body comprises an electrode including a pair of spaced parallel electrode plates, a cavity, and a well disposed inside the electrode plates. The cuvette is configured to produce an electric field between the electrode plates; wherein the electric field is operable to create pores in a cell sample present in the well.

The cuvettes of the present invention are advantageous, especially in electroporation devices. These and additional objects and advantages provided by the cuvettes of the present invention will be more fully understood in view of the following detailed description.

### BRIEF DESCRIPTION OF THE DRAWINGS

The following detailed description of specific embodiments of the present invention can be best understood when read in conjunction with the drawings enclosed herewith. The drawing sheets include:

FIG. 1 is a schematic view of a cuvette according to one or more embodiments of the present invention.

FIG. 2 is a cross sectional view of a cuvette according to one or more embodiments of the present invention.

FIG. 3 is another cross sectional view of a cuvette according to one or more embodiments of the present invention.

FIG. 4 is a front view of a cuvette according to one or more embodiments of the present invention.

FIG. 5 is a top view of an electrode positioned inside the cuvette according to one or more embodiments of the present invention.

FIG. 6 is a further cross sectional view of a cuvette according to one or more embodiments of the present invention.

### DETAILED DESCRIPTION

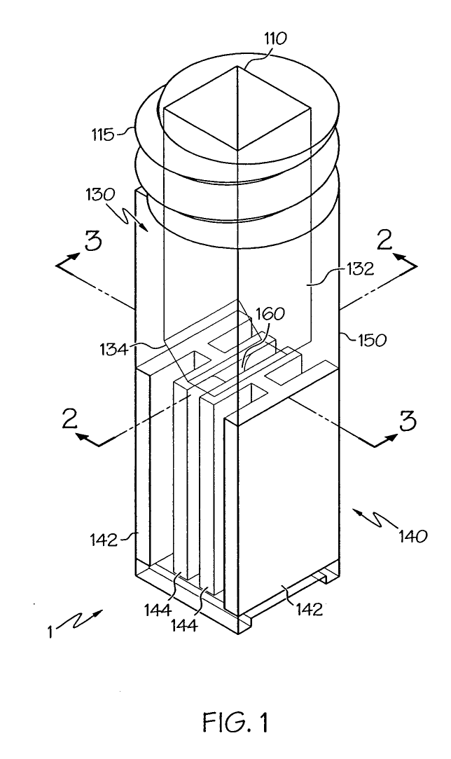

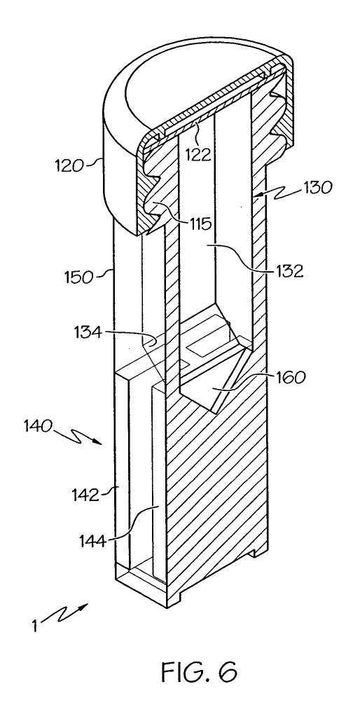

Referring generally to FIGS. 1-6, a cuvette 1 used in electroporation is provided. As shown in FIG. 1, the cuvette 1 comprises an opening 110 at its top. The opening 110 may comprise a threaded portion 115. As shown in FIG. 6, the threading 115 enables a screw top cap 120 to be fastened to the threading 115, thereby sealing the opening 110. The threading 115 and the cap 120 may each comprise plastic, metal, glass, polymeric material, or the like and combinations thereof. In a further embodiment, the cap 120 may comprise an internal seal 122, typically comprising an elastic material, such as rubber and the like. The opening 110 is configured to couple with numerous devices, for example electroporator devices. The opening 110 is square-shaped, but other openings, such as circular shape apertures, are also contemplated. The opening 110 typically may comprise a width of up to about 10 mm, and preferably about 5 mm or less.

Referring to FIG. 1, the cuvette 1 comprises a body 150, which typically defines a rectangular prism with square top and bottom surfaces. Alternatively, the cuvette 1 may comprise a cylindrical structure. The walls of the cuvette body 150 may comprise metal, glass, plastic, polymeric material, or combinations thereof. In one embodiment, the cuvette body 150 comprises polycarbonate.

At the bottom portion of the body 150, the cuvette 1 comprises an electrode 140, typically defined by a pair of spaced parallel electrode walls or plates. The electrode pairs are configured to generate an electric field in the space between the plates. In one embodiment as shown in FIG. 5, the electrode pairs may comprise parallel H-shaped plates. Each pair may also comprise an outer component 142 that extends substantially along at least one wall of the cuvette body 150, so that the electrodes may contact a power source. The pairs may further comprise an inner component 144, wherein the respective inner components 144 are spaced a set distance apart. The electric field is typically generated in the distance between these inner components 144. The electrodes 140 may comprise any suitable conductive material, such as steel or aluminum.

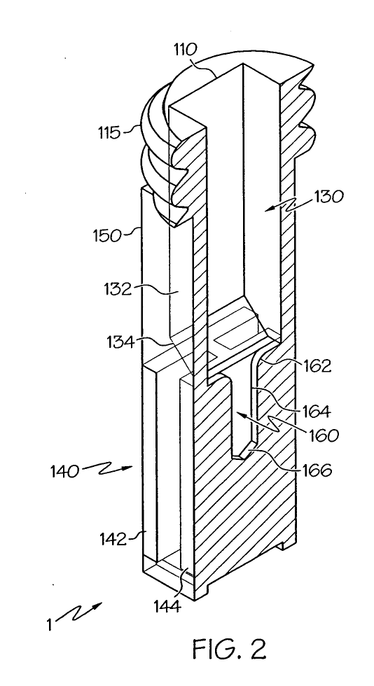

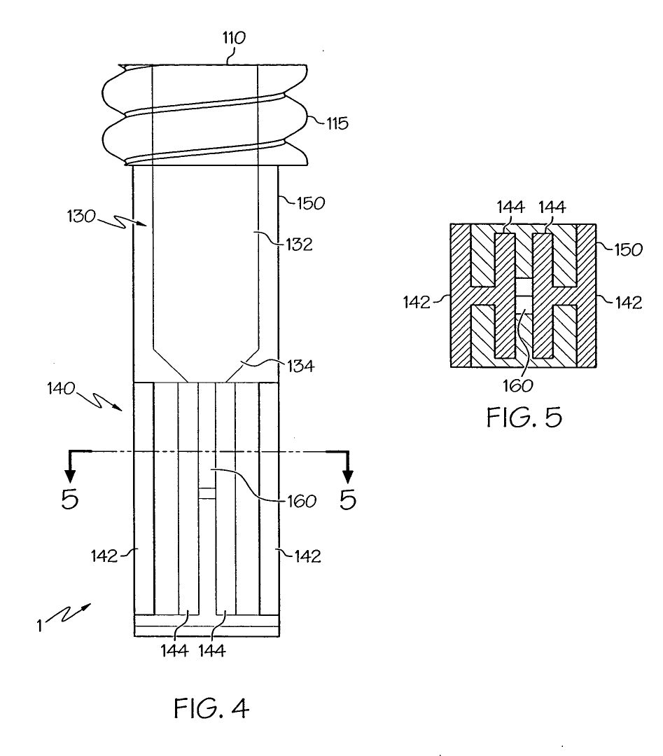

Furthermore, the cuvette 1 also comprises a cavity 130 and a well 160. The cavity 130 is disposed inside the walls of the cuvette body 150, and extends from the opening 110 downwardly to the top of the well 160 disposed between the pair of inner electrode components 144. The cavity 130 generally comprises an upper cavity 132 and a lower cavity 134. As shown in FIG. 4, the upper cavity 132 is generally a straight tube-like section, which extends downwardly from the opening 110 to the lower cavity 134. In a further embodiment, the top section 132 may taper downwardly to the lower cavity 134. As shown in FIGS. 1-4, and 6, the lower cavity 134 is a tapered portion of the cavity 130, which extends from the bottom of the upper cavity 132 to the top of the well 160, which is substantially disposed between the inner electrode components 142 as shown in FIGS. 2 and 5. Typically, as illustrated in FIGS. 1-3, and 6, the width of the well 160 is narrower than the width of the upper cavity 132. The design of the cuvette well 160 is such that its volume can be well below about 100 μL and is typically between about 25 μL to about 40 μL. It is noted that the volume of the well can be changed by modifying the width or depth of the well 160.

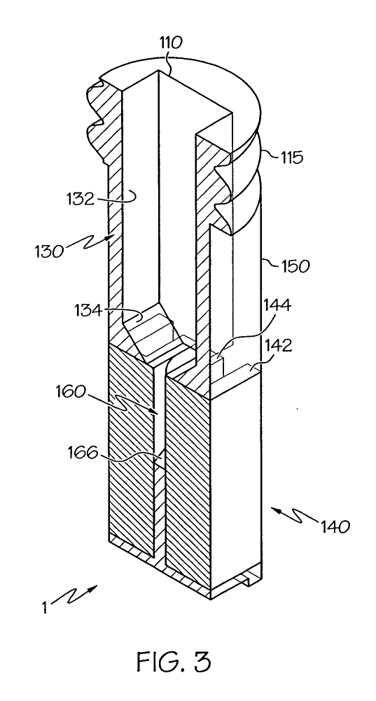

The well 160 may define numerous shapes and configurations. In one embodiment as shown in FIG. 2, the well 160 may comprise a curved portion 162 joined to a straight section 164, which is further connected to a well floor 166. The curved portions of the well 160 are rounded to encourage the transfer of fluid into the well 160. Referring to FIGS. 2 and 3, the well floor 166 may define a triangular configuration, or alternatively a rounded or squared configuration. In another embodiment as shown in FIG. 6, the well 160 may define a V-shaped configuration. Typically, the cavity 130 and the well 160 are comprised of a glass, plastic, metal, polymeric material, or combinations thereof.

The cuvettes 1, described herein, are operable to be used in various electroporation methods and techniques. For example, a cell sample and an implant mixture may be added to the cuvette 1 through the opening 110. The cell sample and implant mixture collect and are mixed in the cuvette well 160 disposed between the inner electrode components 144. Subsequently, the electrodes 140 apply an electrical field to the cell/implant mixture. The electric field creates pores inside the cells, whereupon molecules of the implant agent are inserted into the cells.

In another embodiment, the cuvette 1 may be an operable medium for storing and/or shipping liquids, such as cell samples, inside the cuvette 1. In a specific embodiment, the cuvette 1 may be operable to store cell samples in a frozen state. When the cells are frozen, the cells are biologically inert and can be preserved for years; as a result, the cuvette 1, cap 120 and seal 122 must comprise robust material compositions sufficient to withstand and function at low temperatures. In one embodiment, the cuvette 1 is operable to store cell samples at temperatures as low as about −200° C. In another embodiment, the cuvette 1 is operable at temperatures below about −20° C. In yet another embodiment, the cuvette 1 may operate at temperatures below about −80° C.

In addition to robustness, the electroporation requires sterility because contaminants and particulates can adversely affect cell poration, and/or cell implantation. Accordingly, the cuvette body 150 and cap 120 comprise materials operable to prevent contaminants and particulates from entering the cuvette 1. The seal 122 may provide additional support by preventing possible leakage of liquids out of the cuvette 1 and providing further protection against contaminants. Moreover, cuvettes 1 may also be sterilized by gamma irradiation to eliminate any possible contaminants in the cuvette 1.

It is noted that terms like “specifically,” “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

1. A cuvette including one or more of the novel features described in the present application.

2. A cuvette substantially as described in the specification and in the accompanying drawings.
